(19) United States
(12) Patent Application Publication
(10) Pub. No.: US 2019/0335998 A1
(43) Pub. Date: Nov. 7, 2019

(54) IMPLANTABLE DEVICE FOR PROSTHESIS MONITORING

(71) Applicant: GIOMI Innovation and Research S.r.l., Roma (RM) (IT)

(72) Inventor: Carlo CARUSO, Rome (RM) (IT)

(73) Assignee: GIOMI Innovation and Research S.r.l., Roma (RM) (IT)

(21) Appl. No.: 16/481,116

(22) PCT Filed: Jan. 31, 2017
(86) PCT No.: PCT/IB2017/050509

§ 371 Date: Jul. 26, 2019

(51) Int. Cl. A61B 5/00; A61B 5/01

(52) U.S. Cl. CPC A61B 5/0008; A61B 5/0031; A61B 2562/0271; A61B 5/6862; A61B 5/6876; A61B 5/01

(57) ABSTRACT

An implantable device, suitable to detect and to monitor infectious processes, in particular at a prosthesis is provided. The device has sensor means, apt to detect an index parameter of an infectious process and a control unit connected thereto, the latter being equipped with a storage area for storing the index parameter. The device further has an antenna, connected to the control unit and configured to allow a bidirectional electromagnetic communication with a remote reader of the device. The device is encapsulated by a casing made of elastomeric material.

### TECHNICAL FIELD OF THE INVENTION

The present invention relates to the field of the biomedical devices, in particular to the implantable biomedical devices.

The present invention, more in particular, relates to an implantable device for detecting and monitoring infectious processes at heterologous prostheses.

### BACKGROUND

The most recent implantable devices typically are devices which make use of RFID (Radio Frequency Identification) technology, or a technology which in the main features, thanks to the use of particular electronic labels, allows to obtain information from the implanted device through a remote query of the latter.

In the biomedical field, wherein such devices are intended to work in biological environment, their electronics on board generally is protected by a casing which is made of biocompatible material.

The material used with greater frequency is of ceramic type, for example quartz or silicon, since it is very stable and it has no permeability problems above all in case of devices implanted for a long period of time. However, the selection of ceramic materials involves limited elastic properties of the device structure, above all in terms of possible fastening at implanted prostheses and resistance to mechanical stresses.

A known solution, alternative to the ceramic materials, is the use of polymers, therethrough it is possible to encapsulate completely the implanted device. However, the selection of the type of polymeric material to be used requires great care both due to the permeability thereof and as they are materials prone to degradation phenomena.

In fact, mechanisms degrading the polymers may result from the alteration of the properties thereof and from the consequent release of degradation products which develop an adverse biological response in the tissues surrounding the implanted device.

Apart from possible risks in terms of irreversible damages to the organism wherein the device is implanted, the degradation of the polymers may further compromise the correct detection of physiological parameters potentially of interest in the implant area.

### BRIEF DESCRIPTION OF THE INVENTION

The technical problem placed and solved by the present invention is then to overcome the above-illustrated problems, and this is obtained through an implantable device as defined in claim 1.

In particular, the object of the present invention is to provide an implantable device which is structurally simple, it can be applied in a versatile way and it has high biocompatibility features.

An object of the present invention is to have an implantable device which is capable of detecting and monitoring the possible occurrence of infectious processes at heterologous prostheses.

Additional preferred features of the present invention are defined in the depending claims.

The present invention relates to an implantable device, suitable to detect and to monitor infectious processes, in particular at a prosthesis.

The device comprises sensor means, apt to detect an index parameter of an infectious process and a control unit connected thereto, the latter being equipped with a storage area for storing the above-mentioned index parameter.

The device further comprises an antenna, connected to the control unit and configured to allow a bidirectional electromagnetic communication with a remote reader of the device.

In particular the device is encapsulated by a casing made of elastomeric material.

Such solution allows reducing the risk of processes degrading the device casing, with the advantage of limiting modifications of the state of the tissues surrounding it, to the purpose of a correct evaluation of the index parameter of interest in the prosthesis area.

Still, the use of elastomers as material encapsulating the device allows to give resistance property to cutting, lengthening, abrasion and impermeability property, as well as great elasticity to the latter, to the benefit of the application versatility inside the human body, above all at prostheses.

According to another new aspect, the present invention is addressed to an implantable device, suitable to detect and to monitor infectious processes, in particular at a prosthesis.

The device comprises sensor means, apt to detect an index parameter of an infectious process and a control unit connected thereto, the latter being equipped with a storage area for storing the above-mentioned index parameter.

The device further comprises an antenna, connected to the control unit and configured to allow a bidirectional electromagnetic communication with a remote reader of the device.

In particular the device is encapsulated by a casing and the index parameter detected by the sensor means is the temperature quantity.

Such solution allows the shown device to detect possible variations of temperature values surrounding the prosthesis—and not associated to a systemic increase in the temperature of the body housing the prosthesis—as expression of a phlogistic process affecting the latter.

Then, the possibility of detecting in advance such process is advantageous as it allows to stress for example an advanced antibiotic treatment capable of treating conservatively the prosthetic infection or of intervening surgically in the explant of the prosthesis before dangerous and irreversible events occur.

Advantageously, preferred embodiments of the invention provide that the casing of the device is made of elastomeric material.

The use of elastomeric material allows to reduce the risk of processes degrading the device casing, with the advantage of limiting modifications in the state of the tissues surrounding it, to the benefit of a correct evaluation of the temperature in the area surrounding the prosthesis.

Other advantages, together with the features and the use modes of the present invention will result evident from the following detailed description of preferred embodiments thereof, shown by way of example and not for limitative purpose.

### BRIEF DESCRIPTION OF THE FIGURES

The drawings shown in the enclosed figures will be referred to, wherein:

FIG. 1 shows a schematic representation of the operation of an implantable device, under condition assembled on a prosthesis, according to the present invention;

FIGS. 2A, 2B and 2C show respectively a view from top, a view along a side section and a view along a front section of a first structural detail of a reader to be used in association with the implantable device according to the present invention;

FIGS. 3A, 3B and 3C show respectively a view from top, a view along a side section and a view along a front section of a second structural detail of a reader to be used in association with the implantable device according to the present invention.

### DETAILED DESCRIPTION OF EMBODIMENTS OF THE INVENTION

The present invention will be described hereinafter by referring to the above-mentioned figures.

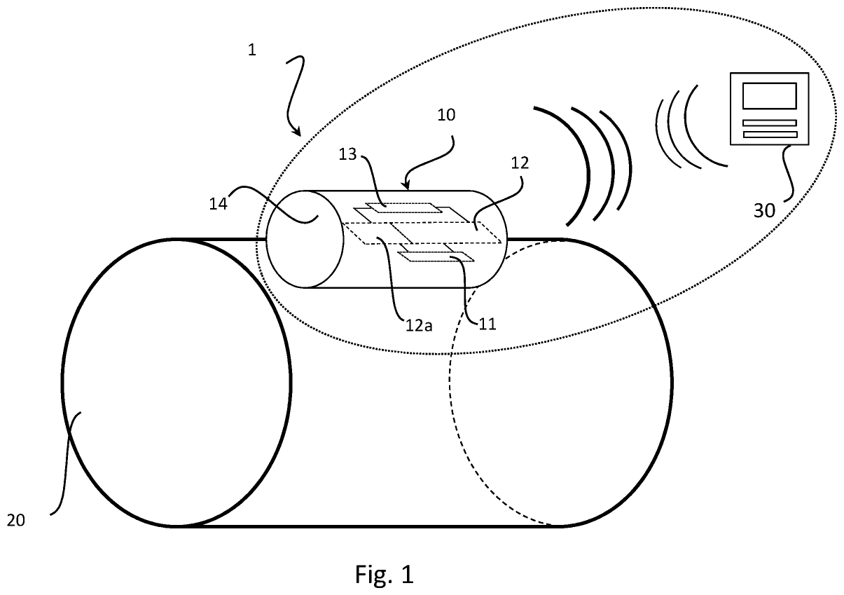

By referring to FIG. 1, a schematic representation is illustrated by way of example and not for limitative purposes, under operating conditions of an implantable device 10 according to the present invention.

Such device 10 preferably is applied in the biomedical field and it allows, in the preferred intended use and as it will be described hereinafter in details, to detect and to monitor the occurrence of possible infectious processes caused by the prosthesis implant inside the human body.

Preferably, the device 10 according to the present invention, is addressed to the use in association to a vascular prosthesis of heterologous type for treating pathologies at the abdominal aorta's charge.

By purely descriptive and not limiting purposes, FIG. 1 then relates to a prosthesis 20 having a substantially hollow cylindrical geometry, and hereinafter in the description one will assume that such prosthesis is made of polymers biocompatible for such application, preferably Dacron® (polyethylene terephthalate), or alternatively ePTFE (polytetrafluoroethylene), which in this field represent the commercially most widespread types.

As it is known, the use of Dacron® preferably will relate to vascular prostheses having medium-big gauges, that is diameters varying from a minimum of about 10 mm until about 20 mm, whereas in case of using ePTFE prostheses having smaller gauges will be referred to, that is comprised between about 4 mm and 8 mm.

In more general terms, and as it can be seen in FIG. 1, the implantable device 10 comprises sensor means 11, apt to detect an index parameter of an infectious process, connected to a control unit 12.

The control unit 12 is equipped with a storage memory, designated with the reference number 12a, for storing the index parameter.

According to a new aspect of the present invention, advantageously the sensor means 11 of the implanted device 10 comprises a temperature sensor configured so as to detect and to monitor a temperature value localized near the prosthesis 20.

Then, one will appreciate that the shown device 10 is capable of detecting possible variations in temperature values surrounding the prosthesis 20—and not associated to a systemic increase in the temperature of the body housing the prosthesis—as expression of a phlogistic process affecting the latter.

Then, the possibility of detecting in advance such process is advantageous since it allows to stress for example an advance antibiotic treatment capable of treating conservatively the prosthetic infection, or to intervene surgically in the explant of the prosthesis before dangerous and irreversible events occur.

To this purpose the control unit 12 is connected to an antenna 13 configured to allow a bidirectional electromagnetic communication with a remote reader 30 which can be associated to the device 10.

In this way the device 10, queried by the reader 30, transmits the detected index parameter, preferably a temperature value, to allow a continuous monitoring of the state of the tissues surrounding the prosthesis 20.

The reader 30 of the device 10 will be now described hereinafter.

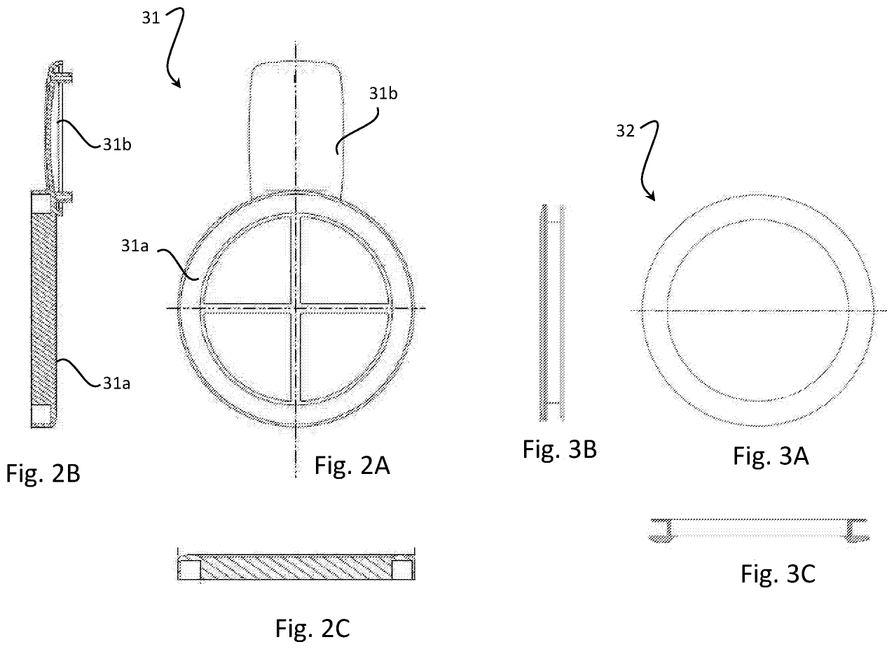

As to what said previously, the reader 30, illustrated by referring to a preferred embodiment in FIGS. 2A-2C and 3A-3C, then comprises means for the bidirectional transmission of electromagnetic fields, apt to cooperate with the device 10, to read the index parameter detected therefrom and, preferably, to feed the device 10 itself.

In the illustrated example, the reader comprises a first 31 and a second 32 component, which can be assembled therebetween and shaped so that the overall configuration thereof allows to house an antenna—not visible in figures.

The reader 30, with its own antenna, reaches the antenna of the device 10 and reads therefrom the value of the index parameter, preferably confirming a maximum reading distance at about 30 cm from the device 10.

Preferably, the antenna of the reader 30 has a circular geometrical shape having a diameter of 12 cm and constituted by 90 turns, and it results to be housed in a seat developing along a portion 31a like a circular crown of the first component 31.

The second component 32 has a geometrical shape analogous to the above-mentioned portion 31a of the first component 31 so as to obtain a shape fixing therebetween and to house the antenna.

As it can be seen in figures, the first component 31 further has a gripping portion 31b containing the electronic components of the reader 30 which result to be connected to the above-mentioned antenna.

Leaving the sofar described structural shape aside, since the application field of the device preferably relates to the monitoring of prostheses implanted in no more hospitalized users, but in users placed in the usual life environment thereof, advantageously the reader 30 is equipped with a system architecture so as to allow an implementation thereof inside devices such as Smartphone or Tablet.

The detecting or monitoring procedure then results to be simple and immediate, as it can be performed by not specialized personnel, in particular by the same person carrying the monitored prosthesis, even if he/she is an old or handicapped person.

To this purpose, the reader 30 preferably is equipped with local peripheral devices for managing the interface with the user, such as for example a key for turning-on, for activating the reading and for turning-off, LEDs for light signalling and multi-tone beepers for acoustic signalling.

In general terms the reader 30 is configured to allow the management of the device 10, the management of the interface with the user, the management of the communication channel for transferring the index parameter and the management of the protocol for communicating with the device 10.

Preferably the latter is a communication channel which makes use of the Bluetooth® standard.

Going back to FIG. 1 and as said previously, the device 10, under operating condition, is implanted inside the human body.

For this reason, it is insulated inside a shell with biocompatibility features. In particular, the device 10 is encapsulated by a casing 14 made of elastomeric material, that is wholly incorporated with the elastomer, so that the latter implements a waterproof casing around the device 10.

One will appreciate that the use of elastomers as material encapsulating the device 10 allows to give resistance property to cutting, lengthening, abrasion and impermeability property, as well as great elasticity to the latter, to the benefit of application versatility inside the human body, above all at prostheses.

In general terms, apart from the features common on themselves between the elastomeric materials, the elastomeric material used to encapsulate the device 10 results to be not transparent, a little viscous and thermally conductive.

Experimental data have confirmed that for the use application of the device 10 according to the present invention, among the elastomeric materials of medical grade, the ones which preferably can be used are those with the commercial name of MDX4-4210 of Dow Corning Corporation.

However other elastomers can be used, such as for example the elastomer known with the commercial name MS 151 AO of Masterbond Inc.

An advantageous aspect of the use of elastomers is further to reduce the risk of processes degrading the casing of the device 10, with the advantage of limiting modifications of the status of the tissues surrounding it, to the purpose of a correct evaluation of the index parameter of interest in the prosthesis area.

The above-mentioned advantages appear particularly when the index parameter detected by the device 10 is the temperature quantity.

Moreover, since the device 10 has to communicate with the reader 30, the variation in the detected index parameter has to be preferably as variation in an electric value of the latter.

Said electric value is subsequently changed into information, preferably transmitted through radiofrequency, to the reader 30.

Although in the scheme of FIG. 1 the device 10 is illustrated with a casing having a cylindrical geometrical shape, other geometrical shapes are possible.

For example the device 10 can be encapsulated by a casing having a substantially prismatic geometrical shape wherein the antenna 13 comprises a solenoid wound on ferrite, or alternatively by a casing 14 with a substantially discoid geometrical shape. In the latter case the antenna 13 preferably has a planar development.

In each case, the device 10 is configured so that during reading, the maximum magnetic field, at the geometrical centre of the antenna 13, thereto the body in which it is implanted is subjected is about 144.3 μT.

Additional features implementing the coupling between device 10 and reader 30 relate to the transmission frequency bands, preferably comprised between 119-135 kHz 66 dBμA/m at 10 m, preferably equal to about 125 kHz.

Such frequencies allow to confirm the emission limit of the magnetic field on the human body at about 12.8/duty-cycle A/m, wherein the duty-cycle is the ratio between transmission periods and 360 seconds, in any range of 360 seconds.

More in details, the casing 14 of the device 10 is equipped with an attachment surface configured for the fastening thereof to the prosthesis 20.

In a preferred embodiment of the present invention, such surface is suitable to receive an adhesive which allows to adhere the casing to the prosthesis.

Experimental data have designated cyanoacrylate as the preferred adhesion type for such application, in particular way in combination with a casing 14 implemented with MDX4-4210 and a prosthesis 20 made of Dacron®.

However, different methods for fastening the device 10 to the prosthesis 20 can be implemented, for example by shaping the attachment surface of the casing 14 made of elastomeric material so that the latter performs the fastening the device 10 to the prosthesis 20.

According to alternative embodiments of the device 10 according to the present invention, the sensor means 11 of the latter can provide several sensors, each one configured to detect a respective index parameter and wherein, preferably, at least one of the sensors allows to detect the temperature quantity. Advantageously, for example, the device 10 can be equipped with two sensors, both configured to detect temperature values, in particular configured for detecting temperature values at different areas of the tissues surrounding the prosthesis 20.

By adding several temperature sensors, enabled for example cyclically, it is possible to determine a temperature gradient, useful when the index parameter to be evaluated is the temperature difference between two tissues, for example infected and not infected portion around the prosthesis. In this way the index parameter can be made independent from the body temperature.

In additional variants, in combination with a sensor configured to detect a temperature value, the sensor means 11 can detect a position quantity, in particular a shifting quantity, or loading quantities.

Shifting measurements can be obtained both by capacitive route and by inductive route depending upon the extent of the shifting to be detected, for example the removal of the device 10 from the prosthesis 20.

Loading measurements can detect possible overloads on the prosthesis 20 and cause the consequent rupture of the sensor means and then the passage from an electrically conductive state of the device 10 to an insulating state.

According to other embodiments, the device 10 for example can make use of the chipless technology or the inductively coupled UHF technology.

The present invention has been sofar described by referring to preferred embodiments thereof. It is to be meant that each one of the technical solutions implemented in the preferred embodiments, herein described by way of example, could be advantageously combined differently therebetween, to create other embodiments, belonging to the same inventive core as defined in the protection scope of the herebelow reported claims.

1. An implantable device, suitable adapted for detecting and monitoring infectious processes, comprising:
one or more sensor elements, configured to detect a parameter index of an infectious process;
a control unit, connected to said one or more sensor elements and equipped with a storage area for storing said index parameter; and
an antenna, connected to said control unit and configured to allow a bidirectional electromagnetic communication with a remote reader of the device;
wherein said implantable device is encapsulated by a casing made of elastomeric material.

2. The implantable device according to claim 1, wherein said index parameter is temperature.

3. An implantable device, adapted to detect and to monitor infectious processes, comprising:
one or more sensor elements, configured to detect parameter index of an infectious process;
a control unit, connected to said one or more sensor elements and equipped with a storage area for storing said parameter; and
an antenna, connected to said control unit and configured to allow a bidirectional electromagnetic communication with a remote reader of the implantable device;
wherein said implantable device is encapsulated by a casing and wherein said parameter is the temperature.

4. The implantable device according to claim 3, wherein said casing is made of elastomeric material.

5. The implantable device according to claim 1, wherein said casing is equipped with an attachment surface configured for the fastening on a prosthesis.

6. The implantable device according to claim 5, wherein said attachment surface is suitable to receive an adhesive, for fastening said casing to the prosthesis.

7. The implantable device according to claim 1, 5, wherein said attachment surface is shaped to allow, to anchor the implantable device to the prosthesis.

8. The implantable device according to claim 1, wherein said one or more sensor elements comprises at least two sensors, wherein each sensor is configured to detect a respective parameter and wherein a parameter detected by one of said two sensors is temperature.

9. The implantable device according to claim 8, wherein said two sensors cooperate to detect a differential value of temperature at different areas of the tissues surrounding a prosthesis.

10. The implantable device according to claim 3, wherein said parameter is a position quantity or a loading quantity.

11. The implantable device according to claim 1, wherein said casing has a substantially prismatic geometry.

12. The implantable device according to claim 1, wherein said casing has a substantially discoid geometrical shape.

13. A reader to be used in association with an implantable device according to claim 1, comprising means for the bidirectional transmission of electromagnetic fields, adapted to cooperate with said implantable device for reading the parameter detected thereby and optionally for powering the implantable device.

14. A kit for detecting and monitoring infectious processes at implanted prostheses comprising:
an implantable device according to claim 1; and
a reader.

15. A prosthesis implantable in the human body, comprising an implantable device according to claim 1.

16. The prosthesis according to claim 15, which is a vascular prosthesis with substantially hollow cylindrical shape and adapted to treat aortic aneurysms.

17. A surgical method for implanting a device for detecting and monitoring infectious processes due to implanted prosthesis, comprising the steps of:
implanting a prosthesis at the area to be treated;
providing an implantable device according to claim 1; and
applying said implantable device to the prosthesis, with an adhesive to allow the adhesion of an attachment surface of the implantable device to said prosthesis.

18. The implantable device of claim 6, wherein the adhesive is cyanoacrylate.

19. The surgical method of claim 17, wherein the adhesive is cyanoacrylate.
